(19) United States
(12) Patent Application Publication
(10) Pub. No.: US 2022/0395483 A1
(43) Pub. Date: Dec. 15, 2022

(54) COMPOSITION AND METHOD FOR CONVERTING HUMAN GILIAL CELLS INTO NEURONS

(71) Applicant: The Penn State Research Foundation, University Park, PA (US)

(72) Inventors: Gong CHEN, Guangzhou (CN); Jui-Chao YIN, State College, PA (US); Xin WANG, State College, PA (US)

(73) Assignee: The Penn State Research Foundation, University Park, PA (US)

(21) Appl. No.: 17/779,418

(22) PCT Filed: Nov. 25, 2020
(86) PCT No.: PCT/US2020/062295

§ 371 Date: May 24, 2022

Related U.S. Application Data
(60) Provisional application No. 62/939,964, filed on Nov. 25, 2019.

(51) Int. Cl. A61K 31/352; A61K 38/28; A61K 31/375; A61K 31/4545; A61P 25/28

(52) U.S. Cl. CPC A61K 31/352; A61K 38/28; A61K 31/375; A61K 31/4545; A61P 25/28

(57) ABSTRACT

Compositions and methods for reprogramming glial cells into neurons are provided. The method entails using a combination of insulin and forskolin generate neurons. The neurons may be generated by converting glial cells into the neurons. The compositions and methods may also include one or both of Vitamin C (VC) and Crizotinub (Cri). Articles of manufacture are provided which include at least one pharmaceutical formulation, packaging, and printed material providing an indication and/or instruction for using the described compounds.

### CROSS REFERENCE TO RELATED APPLICATIONS

This application claims priority to U.S. provisional patent application No. 62/939,964, filed Nov. 25, 2019, the entire disclosure of which is incorporated herein by reference.

### FIELD

The present disclosure relates generally to compositions and methods for treating neurological conditions and converting glial cells into neurons.

### BACKGROUND

There is an ongoing need for alternative compositions and methods that can be used for prophylaxis and/or therapy of a variety of disorders and conditions where generation of functional neurons is desirable. The present disclosure is applicable to this need.

### SUMMARY

In an embodiment, the disclosure includes a method for generating neurons comprising contacting glial cells with a combination of compounds comprising forskolin and insulin, wherein the combination of forskolin and insulin is sufficient to generate the neurons from the glial cells. In an embodiment the disclosure provides a pharmaceutical composition comprising a combination of forskolin and insulin, and the pharmaceutical composition optionally further comprising one or both of Vitamin C (VC) and Crizotinub (Cri). In an embodiment, the disclosure provides an article of manufacture comprising a pharmaceutical composition comprising a combination of forskolin and insulin, the article of manufacture further comprising printed material providing an indication that a combination is for use in treating a condition associated with a need for functional neurons. Thus, compositions and methods for reprogramming glial cells into neurons are provided. The disclosure demonstrates that a combination of insulin and forskolin is sufficient to generate neurons. A combination of insulin and forskolin is also referred to herein as “IFsk.” In embodiments, the disclosure thus provides a cell-free, virus-free, and expression vector free approach to generation of neurons. In embodiments, generating neurons comprises converting glial cells into neurons.

In embodiments, the compositions may comprise additional agents to enhance the described neuronal generation, such as Vitamin C (VC) and Crizotinub (Cri). Data presented in this disclosure support the in vivo use of the described compound combinations for generating neurons in subjects in need thereof, wherein said need arises from any of a wide array of disorders, conditions, and injuries that create a need for neuronal generation, as further described in the detailed description.

### BRIEF DESCRIPTION OF THE FIGURES

FIG. 1. Forskolin and insulin (IFsk) treatment reprogrammed human astrocytes to neurons. (FIG. 1A-FIG. 1B) Human astrocytes were treated with 0.01% dimethylsulfoxide (DMSO), or insulin (I) and forskolin (Fsk) combination (50 mg/L insulin and 10 μM forskolin) for 12 days continuously. Immunohistochemistry was performed with neuronal markers NEUN (red) and MAP2 (green) at 2 weeks after initial drug treatment. (FIG. 1C) Number of NEUN⁺ cells was quantified in each field (0.4 mm²). Statistical analysis revealed that IFsk together generated a significant number of neurons compared with DMSO control. Unpaired t test, **p<0.01. N=4 batches.

FIG. 2. Crizotinib and Vitamin C addition to IFsk further increased neuronal yield. (FIG. 2A) 12-day treatment of 0.02% DMSO almost generated no NEUN⁺ or MAP2⁺ neurons from human astrocytes. (FIG. 2B) Treatment with 0.25 μM Crizotinib and 50 μg/mL Vitamin C, together with IFsk of human astrocytes resulted in a large number of neurons. (FIG. 2C) Quantification of NEUN+ cells showed that CIFskV together induced 75±8 neurons in each field (0.4 mm²), which was significantly different from the DMSO control group. Unpaired t test, ****p<0.0001. N=3 batches.

### DETAILED DESCRIPTION

Unless defined otherwise herein, all technical and scientific terms used in this disclosure have the same meaning as commonly understood by one of ordinary skill in the art to which this disclosure pertains.

When a grouping of alternatives is presented, any and all combinations of the members that make up that grouping of alternatives is specifically envisioned. For example, if an item is selected from a group consisting of A, B, C, and D, the inventors specifically envision each alternative subjectly (e.g., A alone, B alone, etc.), as well as combinations such as A, B, and D; A and C; B and C; etc. The term “and/or” when used in a list of two or more items means any one of the listed items by itself or in combination with any one or more of the other listed items. For example, the expression “A and/or B” is intended to mean either or both of A and B—i.e., A alone, B alone, or A and B in combination. The expression “A, B and/or C” is intended to mean A alone, B alone, C alone, A and B in combination, A and C in combination, B and C in combination, or A, B, and C in combination.

Every numerical range given throughout this specification includes its upper and lower values, as well as every narrower numerical range that falls within it, as if such narrower numerical ranges were all expressly written herein. When a range of numbers is provided herein, the range is understood to be inclusive of the edges of the range as well as any number between the defined edges of the range. For example, “between 1 and 10” includes any number between 1 and 10, as well as the number 1 and the number 10.

When the term “about” is used in reference to a number, it is understood to mean plus or minus 10%. For example, “about 100” would include from 90 to 110.

The singular terms “a” “an” and “the” are not intended to be limiting and include plural referents unless explicitly stated otherwise or the context clearly indicates otherwise.

Any result obtained using a method or composition described herein can be compared to any suitable reference, such as a known value, or a control sample or control value, suitable examples of which will be apparent to those skilled in the art, given the benefit of this disclosure.

The present disclosure comprises compositions and methods that are designed to convert human glial cells into functional neurons. In embodiments, the method comprises contacting glial cells with an effective amount of a combination of insulin and forskolin. In embodiments, contacting the glial cells with the described combination comprises administering an effective amount of a combination if insulin and forskolin to a subject in need thereof.

Forskolin is commercially available and its structure is known in the art. The structure of forskolin as well as its molecular formula is available under PubChem ID 47936. Cri is commercially available and its structure is known in the art. The structure of Cri as well as its molecular formula is available under PubChem ID 11626560. The disclosure includes pharmaceutically acceptable salts of each of the described compounds.

Insulin is also commercially available and its structure and amino acid sequence of is also well known in the art. In embodiments, the insulin comprises an animal-derived or synthetic form of insulin. In embodiments, the insulin comprises a recombinant human insulin which is commercially available from a variety of sources. In embodiments, the insulin comprises a dimer comprising an A-chain and a B-chain, which are linked together by two disulfide bonds. The A-chain comprises 21 amino acids, while the B-chain comprises 30 residues. Cri is commercially available and its structure is known. In embodiments, insulin and forskolin may be the only two compounds administered to the subject to convert glial cells into neurons. Thus, the disclosure includes in a non-limiting embodiment administering to a subject in need thereof a combination of compounds that consists of insulin and forskolin to produce the described neuronal generation. The disclosure, however, includes administering other compounds. For example, it is demonstrated herein that adding VC and Cri to the insulin and forskolin administration enhances the neuronal generating properties of the insulin and forskolin combination. Thus, in embodiments, the disclosure includes administering to a subject in need thereof a combination that comprises or consists of insulin, forskolin, and one or both of VC and Cri, to produce the described neuronal generation. In embodiments, administration of the described compound combination to a subject in need thereof is expected to result in at least some glial cells in the subject being converted into neurons. In embodiments, administration of the described compound combination to a subject in need thereof is expected to result in at least one glial cell in the subject being converted into neurons. In embodiments, conversion into neurons takes place over a period of approximately 7 to 14 days. In embodiments, conversion into neurons takes place between 7 days and 8 days, between 7 days and 9 days, between 7 days and 10 days, between 8 days and 9 days, between 8 days and 10 days, between 8 days and 11 days, between 9 days and 10 days, between 9 days and 11 days, between 9 days and 12 days, between 10 days and 11 days, between 10 days and 12 days, between 10 days and 13 days, between 11 days and 12 days, between 11 days and 13 days, between 11 days and 14 days, between 12 days and 13 days, between 12 days and 14 days, or between 13 days and 14 days. In embodiments, conversion into neurons does not take more than 7, 8, 9, 10, 11, 12, 13, 14, 15, 16, 17, 18, 19, 20, or 21 days.

In embodiments, the disclosure comprises but is not necessarily limited to generation of new neurons from endogenous glial cells, and can include generating new neurons from glia-like cells created due to injury or a disease condition in the central or peripheral nervous system using indicated compounds, which is expected to be useful for a variety of therapies, non-limiting embodiments of which include brain and spinal cord repair.

The neurons generated using the described compositions and methods are generally functional neurons, and thus are capable, of non-limiting example of, at least one of: synapse formation, axon formation, dendrite formation, or neurotransmitter release. Non-limiting examples of the generated neurons produced by the described methods and compositions are unipolar, bipolar, or multipolar neurons. Non-limiting examples of the neurons comprise any type of neuron, such as basket cells, Lugaro cells, medium spiny neurons, Purkinje cells, or spindle cells. In embodiments, the neurons are selected from the group consisting of cholinergic neurons, GABAergic neurons, glutamatergic neurons, dopaminergic neurons, epinephrinergic neurons, motor neurons, peptidergic neurons, and serotonergic neurons. Thus, in embodiments, glial cells, such as astrocytes are reprogrammed so that they are converted into functional neurons. Functional neurons can exhibit properties which can comprise but are not necessarily limited to firing repetitive action potentials, developing a plurality of dendritic branches, and release of neurotransmitters, including but not necessarily limited to Glutamate (glutamic acid), dopamine, acetylcholine, serotonin, Norepinephrine (noradrenaline), and γ-Aminobutyric acid (GABA). Thus, the disclosure is expected to facilitate development of new cortical forebrain neurons, or midbrain neurons, or hindbrain neurons, or spinal cord neurons, or peripheral neurons, or combinations thereof by using methods and compositions described herein adapted as necessary by those skilled in the art in a manner that will be apparent given the benefit of the present disclosure. In embodiments, glial cells are converted into neurons that express one or both of the neuronal markers NeuN (also referred to as NEUN) or Microtubule-associated protein 2 (MAP-2). In embodiments, the generated neurons express Dcx. In embodiments, the generated neurons do not express glial fibrillary acidic protein (GFAP).

In embodiments, glial cells are reprogrammed to form neurons. The glial cells converted into neurons may be any type of glial cells. In embodiments, glial cells are non-neuronal cells present in the central nervous system, including but not necessarily limited to the brain, and thus, prior to being contacted with the described compound combination, do not produce electrical impulses. In embodiments, the glial cells comprise astrocytes. In embodiments, the astrocytes are protoplasmic or fibrous astrocytes. In embodiments, the glial cells comprise reactive astrocytes, which are the main cellular component of glial scars. In embodiments, the glial cells are NG2 glia. In embodiments, the glial cells are microglia. In embodiments, the glial cells are radial glia. In embodiments, the glial cells express one or more markers that selected from GFAP, Aquaporin-4 (AQP4), glutamine synthetase, 10-formyltetrahydrofolate dehydrogenase (ALDH1L1), and combinations thereof. In embodiments, the glial cells express GFAP. In embodiments, the glial cells expression AQP4. In embodiments, the glial cells express glutamine synthetase. In embodiments, the glial cells express ALDH1L1.

As used herein, the term “subject” refers to any animal subject. Non-limiting examples of animal subjects include humans, laboratory animals (e.g., non-human primates, rats, mice), livestock (e.g., cows, sheep, goats, pigs, turkeys, chickens), and household pets (e.g., dogs, cats, rodents, etc.).

In embodiments, the disclosure is expected to be broadly applicable for therapy of any human subject in need of neuronal generation. In embodiments, a subject in need of neuronal generation has a neurological condition. The need for neuronal generation arises as a consequence of any of a variety of conditions, disorders or injuries that affect neuronal function, and/or reduce the number of functional neurons in the subject. Thus, the disclosure is applicable to prophylaxis and/or therapy of conditions which include but are not necessarily limited to ischemic brain damage, such as that caused by stroke, hypoxia or other brain trauma, or glial scarring, or neurodegeneration, or aging, or microcephaly, or severe seizure that causes neuronal loss. In embodiments, a neurological condition is selected from the group consisting of Alzheimer's disease, Parkinson's disease, amyotrophic lateral sclerosis (ALS), Huntington's disease, epilepsy, physical injury, stroke, cerebral aneurysm, traumatic brain injury, concussion, a tumor, inflammation, infection, ataxia, brain atrophy, spinal cord atrophy, multiple sclerosis, traumatic spinal cord injury, ischemic or hemorrhagic myelopathy (myelopathy), global ischemia, hypoxic ischemic encephalopathy, embolism, fibrocartilage embolism myelopathy, thrombosis, nephropathy, chronic inflammatory disease, meningitis, and cerebral venous sinus thrombosis.

In embodiments, the disclosure is applicable to treating neurodegenerative disorders in a subject in need thereof. In embodiments, the disclosure is applicable to treating neurological conditions in a subject in need thereof. In embodiments, the subject is in need of treatment for a condition selected from the group consisting of Alzheimer's disease, another conditions which presents with dementia, Chronic Traumatic Encephalopathy (CTE), Parkinson's disease, Huntington's disease, multiple sclerosis, spinal cord injury, spinal muscular atrophy, Amyotrophic lateral sclerosis (ALS), and stroke. In embodiments, the subject has Alzheimer's disease. In embodiments, the subject has a second condition which presents with dementia. In embodiments, the subject has CTE. In embodiments, the subject has Parkinson's disease. In embodiments, the subject has Huntington's disease. In embodiments, the subject has multiple sclerosis. In embodiments, the subject has spinal cord injury. In embodiments, the subject has muscular atrophy. In embodiments, the subject has ALS. In embodiments, the subject has stroke.

In embodiments, a subject in need thereof is a male. In embodiments, a subject in need thereof is a female. In embodiments, a subject in need thereof is gender neutral. In embodiments, a subject in need thereof is a premature newborn. In embodiments, a premature newborn is born before 36 weeks gestation. In embodiments, a subject in need thereof is a term newborn. In embodiments, a term newborn is below about 2 months old. In embodiments, a subject in need thereof is a neonate. In embodiments, a neonate is below about 1 month old. In embodiments, a subject in need thereof is an infant. In embodiments, an infant is between 2 months and 24 months old. In embodiments, an infant is between 2 months and 3 months, between 2 months and 4 months, between 2 months and 5 months, between 3 months and 4 months, between 3 months and 5 months, between 3 months and 6 months, between 4 months and 5 months, between 4 months and 6 months, between 4 months and 7 months, between 5 months and 6 months, between 5 months and 7 months, between 5 months and 8 months, between 6 months and 7 months, between 6 months and 8 months, between 6 months and 9 months, between 7 months and 8 months, between 7 months and 9 months, between 7 months and 10 months, between 8 months and 9 months, between 8 months and 10 months, between 8 months and 11 months, between 9 months and 10 months, between 9 months and 11 months, between 9 months and 12 months, between 10 months and 11 months, between 10 months and 12 months, between 10 months and 13 months, between 11 months and 12 months, between 11 months and 13 months, between 11 months and 14 months, between 12 months and 13 months, between 12 months and 14 months, between 12 months and 15 months, between 13 months and 14 months, between 13 months and 15 months, between 13 months and 16 months, between 14 months and 15 months, between 14 months and 16 months, between 14 months and 17 months, between 15 months and 16 months, between 15 months and 17 months, between 15 months and 18 months, between 16 months and 17 months, between 16 months and 18 months, between 16 months and 19 months, between 17 months and 18 months, between 17 months and 19 months, between 17 months and 20 months, between 18 months and 19 months, between 18 months and 20 months, between 18 months and 21 months, between 19 months and 20 months, between 19 months and 21 months, between 19 months and 22 months, between 20 months and 21 months, between 20 months and 22 months, between 20 months and 23 months, between 21 months and 22 months, between 21 months and 23 months, between 21 months and 24 months, between 22 months and 23 months, between 22 months and 24 months, and between 23 months and 24 months old. In embodiments, a subject in need thereof is a toddler. In embodiments, a toddler is between 1 year and 4 years old. In embodiments, a toddler is between 1 year and 2 years, between 1 year and 3 years, between 1 year and 4 years, between 2 years and 3 years, between 2 years and 4 years, and between 3 years and 4 years old. In embodiments, a subject in need thereof is a young child. In embodiments, a young child is between 2 years and 5 years old. In embodiments, a young child is between 2 years and 3 years, between 2 years and 4 years, between 2 years and 5 years, between 3 years and 4 years, between 3 years and 5 years, and between 4 years and 5 years old. In embodiments, a subject in need thereof is a child. In embodiments, a child is between 6 years and 12 years old. In embodiments, a child is between 6 years and 7 years, between 6 years and 8 years, between 6 years and 9 years, between 7 years and 8 years, between 7 years and 9 years, between 7 years and 10 years, between 8 years and 9 years, between 8 years and 10 years, between 8 years and 11 years, between 9 years and 10 years, between 9 years and 11 years, between 9 years and 12 years, between 10 years and 11 years, between 10 years and 12 years, and between 11 years and 12 years old. In embodiments, a subject in need thereof is an adolescent. In embodiments, an adolescent is between 13 years and 19 years old. In embodiments, an adolescent is between 13 years and 14 years, between 13 years and 15 years, between 13 years and 16 years, between 14 years and 15 years, between 14 years and 16 years, between 14 years and 17 years, between 15 years and 16 years, between 15 years and 17 years, between 15 years and 18 years, between 16 years and 17 years, between 16 years and 18 years, between 16 years and 19 years, between 17 years and 18 years, between 17 years and 19 years, and between 18 years and 19 years old. In embodiments, a subject in need thereof is a pediatric subject. In embodiments, a pediatric subject between 1 day and 18 years old. In embodiments, a pediatric subject is between 1 day and 1 year, between 1 day and 2 years, between 1 day and 3 years, between 1 year and 2 years, between 1 year and 3 years, between 1 year and 4 years, between 2 years and 3 years, between 2 years and 4 years, between 2 years and 5 years, between 3 years and 4 years, between 3 years and 5 years, between 3 years and 6 years, between 4 years and 5 years, between 4 years and 6 years, between 4 years and 7 years, between 5 years and 6 years, between 5 years and 7 years, between 5 years and 8 years, between 6 years and 7 years, between 6 years and 8 years, between 6 years and 9 years, between 7 years and 8 years, between 7 years and 9 years, between 7 years and 10 years, between 8 years and 9 years, between 8 years and 10 years, between 8 years and 11 years, between 9 years and 10 years, between 9 years and 11 years, between 9 years and 12 years, between 10 years and 11 years, between 10 years and 12 years, between 10 years and 13 years, between 11 years and 12 years, between 11 years and 13 years, between 11 years and 14 years, between 12 years and 13 years, between 12 years and 14 years, between 12 years and 15 years, between 13 years and 14 years, between 13 years and 15 years, between 13 years and 16 years, between 14 years and 15 years, between 14 years and 16 years, between 14 years and 17 years, between 15 years and 16 years, between 15 years and 17 years, between 15 years and 18 years, between 16 years and 17 years, between 16 years and 18 years, and between 17 years and 18 years old. In embodiments, a subject in need thereof is a geriatric subject. In embodiments, a geriatric subject is between 65 years and 95 or more years old. In embodiments, a geriatric subject is between 65 years and 70 years, between 65 years and 75 years, between 65 years and 80 years, between 70 years and 75 years, between 70 years and 80 years, between 70 years and 85 years, between 75 years and 80 years, between 75 years and 85 years, between 75 years and 90 years, between 80 years and 85 years, between 80 years and 90 years, between 80 years and 95 years, between 85 years and 90 years, and between 85 years and 95 years old. In embodiments, a subject in need thereof is an adult. In embodiments, an adult subject is between 20 years and 95 or more years old. In embodiments, an adult subject is between 20 years and 25 years, between 20 years and 30 years, between 20 years and 35 years, between 25 years and 30 years, between 25 years and 35 years, between 25 years and 40 years, between 30 years and 35 years, between 30 years and 40 years, between 30 years and 45 years, between 35 years and 40 years, between 35 years and 45 years, between 35 years and 50 years, between 40 years and 45 years, between 40 years and 50 years, between 40 years and 55 years, between 45 years and 50 years, between 45 years and 55 years, between 45 years and 60 years, between 50 years and 55 years, between 50 years and 60 years, between 50 years and 65 years, between 55 years and 60 years, between 55 years and 65 years, between 55 years and 70 years, between 60 years and 65 years, between 60 years and 70 years, between 60 years and 75 years, between 65 years and 70 years, between 65 years and 75 years, between 65 years and 80 years, between 70 years and 75 years, between 70 years and 80 years, between 70 years and 85 years, between 75 years and 80 years, between 75 years and 85 years, between 75 years and 90 years, between 80 years and 85 years, between 80 years and 90 years, between 80 years and 95 years, between 85 years and 90 years, and between 85 years and 95 years old. In embodiments, a subject in need thereof is between 1 year and 5 years, between 2 years and 10 years, between 3 years and 18 years, between 21 years and 50 years, between 21 years and 40 years, between 21 years and 30 years, between 50 years and 90 years, between 60 years and 90 years, between 70 years and 90 years, between 60 years and 80 years, or between 65 years and 75 years old. In embodiments, a subject in need thereof is a young old subject (65 to 74 years old). In embodiments, a subject in need thereof is a middle old subject (75 to 84 years old). In embodiments, a subject in need thereof is an old subject (>85 years old).

In embodiments, a subject treated according to the method of the disclosure is not being treated with insulin for a condition other than for a need for neuronal generation. In embodiments, the subject does not have Type I or Type II diabetes. In embodiments, a subject treated according to the method of the disclosure is being treated with insulin for a condition other than for a need for neuronal generation. In embodiments, the subject does have Type I or Type II diabetes.

In embodiments, the subject is in need of the described therapy as a result of injury, which can result from a number of causes that are known in the art, and which typically involve astrogliosis after injury or disease processes in the central nervous system including brain and spinal cord, and peripheral nervous system.

In general, methods of the disclosure comprise administering an effective amount of the compounds described herein to a subject such that the number of neurons in the subject is increased. The compounds can be administered in amounts that are the same or similar to those for which FDA approval is already in place. Dosages for each of the FDA approved drugs can be found, for example, in www.accessdata.fda.gov/scripts/cder/drugsatfda/, the disclosure of which that pertains to the described compounds is incorporated herein by reference it exists on the effective filing date of this application or patent. Thus, appropriate dosing of the compound(s) can be determined in conjunction with the knowledge of the skilled artisan, given the benefit of the present disclosure. In embodiments, the weight and age of the subject, personal history of neuronal damage or disease and risk for experiencing same neuronal damage, or the presence of glial scarring or reactive gliosis, may be taken into account when determining an effective amount of the active ingredient and dosing regimen. In embodiments the compounds are administered in an amount of about 0.01 nmol to about 500 nmol a day, inclusive, and including all integers and ranges there between, depending on which delivering method being used. In embodiments, the compounds are administered in an amount of about 0.01 nmol to about 25 nmol, about 0.01 nmol to about 50 nmol, about 0.01 nmol to about 75 nmol, about 25 nmol to about 50 nmol, about 25 nmol to about 75 nmol, about 25 nmol to about 100 nmol, about, about 50 nmol to about 75 nmol, about 50 nmol to about 100 nmol, about 50 nmol to about 125 nmol, about 75 nmol to about 100 nmol, to about 75 nmol to about 125 nmol, to about 75 nmol to about 150 nmol, to about 100 nmol to about 125 nmol to about 100 nmol to about 150 nmol, to about 100 nmol to about 175 nmol, 125 nmol to about 150 nmol, about 125 nmol to about 175 nmol, about 125 nmol to about 200 nmol, about 150 nmol to about 175 nmol, about 150 nmol to about 200 nmol, about 150 nmol to about 225 nmol, about 175 nmol to about 200 nmol, to about 175 nmol to about 225 nmol, to about 175 nmol to about 250 nmol, to about 200 nmol to about 225 nmol to about 200 nmol to about 250 nmol, to about 200 nmol to about 275 nmol, 225 nmol to about 250 nmol, about 225 nmol to about 275 nmol, about 225 nmol to about 300 nmol, about 250 nmol to about 275 nmol, about 250 nmol to about 300 nmol, about 250 nmol to about 325 nmol, about 275 nmol to about 300 nmol, to about 275 nmol to about 325 nmol, to about 275 nmol to about 350 nmol, to about 300 nmol to about 325 nmol to about 300 nmol to about 350 nmol, to about 300 nmol to about 375 nmol, 325 nmol to about 350 nmol, about 325 nmol to about 375 nmol, about 325 nmol to about 400 nmol, about 350 nmol to about 375 nmol, about 350 nmol to about 400 nmol, about 350 nmol to about 425 nmol, about 375 nmol to about 400 nmol, to about 375 nmol to about 425 nmol, to about 375 nmol to about 450 nmol, to about 400 nmol to about 325 nmol to about 400 nmol to about 450 nmol, to about 400 nmol to about 475 nmol, to about 425 nmol to about 450 nmol, to about 425 nmol to about 475 nmol, to about 425 nmol to about 500 nmol, to about 450 nmol to about 475 nmol, or to about 450 nmol to about 500 nmol day.

In embodiments, the compounds are provided in a single, multiple, or controlled release dose regimen. In embodiments, the compounds are administered concurrently. In embodiments, the compounds are administered sequentially. In embodiments, all of at least two of the compounds are administered as a component of the same pharmaceutical formulation. In embodiments, the only active ingredients in the combination are forskolin and insulin. In embodiment, the only active ingredients in the combination are forskolin and insulin, and at least one of VC or Cri. In embodiments, the only active ingredients in the combination are forskolin insulin, VC and Cri.

“Active ingredient” means a compound that acts on the glial cells to convert the glial cells to become neurons. Thus, “active ingredient” does not include agents that are added to, for example, a pharmaceutical formulation to contain or otherwise facilitate delivery of the active ingredients to the glial cells, such agents including but not necessarily limited to buffers, salts, pharmaceutically suitable excipients, carriers, and the like. Accordingly, in embodiments, the disclosure includes pharmaceutical formulations and methods of using said formulations wherein the only active ingredients in the pharmaceutical formulations consists of insulin and forskolin, insulin, forskolin and VC, or insulin, forskolin, VC and Cri. Nevertheless, in embodiments, the described method can be combined with other suitable neuronal generation compositions and methods provided they are free of cellular and recombinant polynucleotide compositions.

In embodiments, the term “therapeutically effective dose” or pharmaceutically active dose” refers to an amount of insulin, forskolin, VC, and Cri, either alone or in combination that converts glial cells to neurons. In embodiments, the therapeutically effective dose treats a neurological condition.

In embodiments, the therapeutically effective dose of insulin is between 5 mg/mL and 100 mg/mL. In embodiments, the therapeutically effective dose of insulin is between 5 mg/mL and 10 mg/mL, 5 mg/mL and 15 mg/mL, between 5 mg/mL and 20 mg/mL, between 10 mg/mL and 15 mg/mL, between 10 mg/mL and 20 mg/mL, between 10 mg/mL and 25 mg/mL, between 15 mg/mL and 20 mg/mL, between 15 mg/mL and 25 mg/mL, between 15 mg/mL and 30 mg/mL, between 20 mg/mL and 25 mg/mL, between 20 mg/mL and 30 mg/mL, between 20 mg/mL and 35 mg/mL, between 25 mg/mL and 30 mg/mL, between 25 mg/mL and 35 mg/mL, between 25 mg/mL and 40 mg/mL, between 30 mg/mL and 35 mg/mL, between 30 mg/mL and 40 mg/mL, between 30 mg/mL and 45 mg/mL, between 35 mg/mL and 40 mg/mL, between 35 mg/mL and 45 mg/mL, between 35 mg/mL and 50 mg/mL, between 40 mg/mL and 45 mg/mL, between 40 mg/mL and 50 mg/mL, between 40 mg/mL and 55 mg/mL, between 45 mg/mL and 50 mg/mL, between 45 mg/mL and 55 mg/mL, between 45 mg/mL and 60 mg/mL, between 50 mg/mL and 55 mg/mL, between 50 mg/mL and 60 mg/mL, between 50 mg/mL and 65 mg/mL, between 55 mg/mL and 60 mg/mL, between 55 mg/mL and 65 mg/mL, between 55 mg/mL and 70 mg/mL, between 60 mg/mL and 65 mg/mL, between 60 mg/mL and 70 mg/mL, between 60 mg/mL and 75 mg/mL, between 65 mg/mL and 70 mg/mL, between 65 mg/mL and 75 mg/mL, between 65 mg/ml and 80 mg/mL, between 70 mg/mL and 75 mg/mL, between 70 mg/mL and 80 mg/mL, between 70 mg/mL and 85 mg/mL, between 75 mg/mL and 80 mg/mL, between 75 mg/mL and 85 mg/mL, between 75 mg/mL and 90 mg/mL, between 80 mg/mL and 85 mg/mL, between 80 mg/mL and 90 mg/mL, between 80 mg/mL and 95 mg/mL, between 85 mg/mL and 90 mg/mL, between 85 mg/mL and 95 mg/mL, between 85 mg/mL and 100 mg/mL, between 90 mg/mL and 95 mg/mL, between 90 mg/mL and 100 mg/mL, or between 95 mg/mL and 100 mg/mL.

In embodiments, the therapeutically effective dose of forskolin is between 0.1 μM and 20 μM. In embodiments, the therapeutically effective dose of forskolin is between 0.1 μM and 1 μM, between 0.1 μM and 5 μM, between 0.1 μM and 10 μM, between 1 μM and 5 μM, between 1 μM and 10 μM, between 1 μM and 15 μM, between 5 μM and 10 μM, between 5 μM and 15 μM, between 50 μM and 20 μM, between 10 μM and 15 μM, between 10 μM and 20 μM, or between 15 μM and 20 μM.

In embodiments, the therapeutically effective dose of Cri is between 0.1 μM and 1 μM. In embodiments, the therapeutically effective dose of Cri is between 0.1 μM and 0.2 μM, between 0.1 μM and 0.3 μM, between 0.1 μM and 0.4 μM, between 0.2 μM and 0.3 μM, between 0.2 μM and 0.4 μM, between 0.2 μM and 0.5 μM, between 0.3 μM and 0.4 μM, between 0.3 μM and 0.5 μM, between 0.3 μM and 0.6 μM, between 0.4 μM and 0.5 μM, between 0.4 μM and 0.6 μM, between 0.4 μM and 0.7 μM, between 0.5 μM and 0.6 μM, between 0.5 μM and 0.7 μM, between 0.5 μM and 0.8 μM, between 0.6 μM and 0.7 μM, between 0.6 μM and 0.8 μM, between 0.6 μM and 0.9 μM, between 0.7 μM and 0.8 μM, between 0.7 μM and 0.9 μM, between 0.7 μM and 1.0 μM, between 0.8 μM and 0.9 μM, between 0.8 and 1.0 μM, or between 0.9 μM and 1 μM.

In embodiments, the therapeutically effective dose of VC is between 1 μg/mL and 10 μg/mL. In embodiments, the therapeutically effective dose of VC is between 1 μg/mL and 2 μg/mL, between 1 μg/mL and 3 μg/mL, between 1 μg/mL and 4 μg/mL, between 2 μg/mL and 3 μg/mL, between 2 μg/mL and 4 μg/mL, between 2 μg/mL and 5 μg/mL, between 3 μg/mL and 4 μg/mL, between 3 μg/mL and 5 μg/mL, between 3 μg/mL and 6 μg/mL, between 4 μg/mL and 5 μg/mL, between 4 μg/mL and 6 μg/mL, between 4 μg/mL and 7 μg/mL, between 5 μg/mL and 6 μg/mL, between 5 μg/mL and 7 μg/mL, between 5 μg/mL and 8 μg/mL, between 6 μg/mL and 7 μg/mL, between 6 μg/mL and 8 μg/mL, between 6 μg/mL and 9 μg/mL, between 7 μg/mL and 8 μg/mL, between 7 μg/mL and 9 μg/mL, between 7 μg/mL and 10 μg/mL, between 8 μg/mL and 9 μg/mL, between 8 and 1.0 μg/mL, or between 9 μg/mL and 1.0 μg/mL.

In embodiments, the therapeutically effective dose is delivered to subject in need at least once daily or at least once weekly for at least two consecutive days or weeks. In embodiments, therapeutically effective dose is delivered to subject in need thereof at least once daily or at least once weekly for at least 3, 4, 5, 6, 7, 8, 9, 10, 11, 12, 13, 14, or 15 consecutive days or weeks. In embodiments, therapeutically effective dose is delivered to subject in need thereof at least once daily or at least once weekly for at least 1, 2, 3, 4, 5, 6, 7, 8, 9, 10, 11, or 12 consecutive weeks. In embodiments, therapeutically effective dose is delivered to subject in need thereof at least once daily or at least once weekly for at most 4, 5, 6, 7, 8, 9, 10, 11, 12, 13, 14, 15, 16, 17, 18, 19, or 20 consecutive days or weeks. In embodiments, therapeutically effective dose is delivered to subject in need thereof at least once daily or at least once weekly for at most 1, 2, 3, 4, 5, 6, 7, 8, 9, 10, 11, or 12 consecutive weeks or months. In embodiments, therapeutically effective dose is delivered to subject in need thereof is administered at least once for at least 1, 2, 3, 4, 5, 6, 7, 8, 9, 10, 11, or 12 consecutive months or years, chronically for a subject's entire life span, or an indefinite period of time. In embodiments, therapeutically effective dose is delivered to subject in need thereof once a year for 2 consecutive years, 3 consecutive years, or 5 consecutive years. In embodiments, therapeutically effective dose is delivered to subject in need thereof once a year for 2 consecutive years. In embodiments, therapeutically effective dose is delivered to subject in need thereof once a year for 3 consecutive years. In embodiments, therapeutically effective dose is delivered to subject in need thereof once a year for 5 consecutive years.

In embodiments, a composition provided herein, consists essentially of forskolin and insulin. In embodiments, a composition provided herein, consists essentially of forskolin, insulin, and VC. In embodiments, a composition provided herein, consists essentially of forskolin, insulin, VC, and Cri.

Compositions comprising the compounds of this disclosure can be provided in pharmaceutical formulations. The form of pharmaceutical preparation is not particularly limited, but generally comprises these active ingredients and at least one inactive ingredient. In embodiments suitable pharmaceutical compositions can be prepared by mixing any one or combination of the compounds with a pharmaceutically-acceptable carrier, diluent or excipient, and suitable such components are well known in the art. Some examples of such carriers, diluents and excipients can be found in: Remington: The Science and Practice of Pharmacy (2005) 21st Edition, Philadelphia, Pa. Lippincott Williams & Wilkins. In embodiments, the pharmaceutical formulations are suitable for delivering the active ingredients across the blood-brain barrier, and/or to the spinal cord or other components of the central nervous system. Such compositions can comprise, for example, lipid formulations or other nano-particle based delivery systems.

In embodiments, the pharmaceutical formulation is suitable for oral administration, and thus can be provided in an aerosolized, liquid or solid dosage form. Solid dosage forms include but are not necessarily limited to tablets, capsules, caplets, and strips, for swallowing or oral dissolution, and may be provided for rapid or extended release, or to release distinct compounds in a desirable series over a period of time. Separate pharmaceutical compositions comprising one or any combination of the compounds can also be used.

With respect to the administration of the pharmaceutical formulations, the route of administration can be any suitable route. In embodiments, the composition comprising the compound(s) is delivered orally. In other non-limiting embodiments, the composition is administered intravenously, parenterally, subcutaneously, intraperitoneally, transdermally, by intranasal instillation, by implantation, intraarterially, or by intrathecal administration. In embodiments, an implantable medical device can be used, such as a pump, including but not limited to an osmotic pump. In embodiments the compositions comprising the compounds is delivered via an intracranial route.

In embodiments, the disclosure includes nutraceutical compositions, which are designed to impart to a subject a beneficial effect that is related to improved neuronal health and/or function. In certain embodiments, the described compositions can be used to improve the general well-being of a subject, or the cognitive capability of a subject, such as for improved memory or maintenance of memory. In embodiments the compositions are useful for improving any or all of short term memory, long term memory, or motor skills, including but not necessarily limited to gross and fine motor skills. Thus, use of nutritional supplements comprising the small molecules described herein are encompassed by this disclosure.

In embodiments, the therapeutically effective dose of this disclosure, achieves a remission, cure, response rate, or resolution rate of a neurological condition of at least about 50%. In embodiments, a therapeutically effective dose eliminates, reduces, slows, or delays, one or more neurological condition symptoms. Non-limiting examples of neurological condition symptoms include tremor, slowed movement (bradykinesia), rigid muscles, impaired posture and balance, loss of automatic movements, uncoordinated movement, uncontrolled movement, spontaneous jerking movement, speech changes, numbness, and writing changes. In embodiments, a neurological condition symptom is a movement symptom. Non-limiting examples of movement symptoms include impairment of an involuntary movement or an impairment of a voluntary movement. In embodiments, a neurological condition symptom is a cognitive symptom. Non-limiting examples of cognitive symptoms include fine motor skills, tremors, seizures, chorea, dystonia, dyskinesia, slow or abnormal eye movements, impaired gait, impaired posture, impaired balance, difficulty with speech, difficulty with swallowing, difficulty organizing, difficulty prioritizing, difficulty focusing on tasks, lack of flexibility, lack of impulse control, outbursts, lack of awareness of one's own behaviors and/or abilities, slowness in processing thoughts, difficulty in learning new information, difficulty in remember things, difficulty in communications, difficulty in following orders, difficulty in executing tasks.

In embodiments, neurological condition symptom is a psychiatric symptom. Non-limiting examples of psychiatric symptoms include depression, irritability, sadness or apathy, social withdrawal, insomnia, fatigue, lack of energy, obsessive-compulsive disorder, mania, bipolar disorder, and weight loss. In embodiments, a neurological condition symptom is at least one damaged blood vessel. In embodiments, a neurological condition symptom is a damaged blood brain barrier. In embodiments, a neurological condition symptom is damaged blood flow. Non-limiting examples of tests to evaluate the elimination, reduction, slow, or delay, of neurological condition symptoms include the unified Huntington's disease rating scale (UHDRS) score, UHDRS Total Functional Capacity (TFC), UHDRS Functional Assessment, UHDRS Gait score, UHDRS Total Motor Score (TMS), Hamilton depression scale (HAM-D), Columbia-suicide severity rating scale (C-SSRS), Montreal cognitive assessment (MoCA), modified Rankin Scale (mRS), National Institutes of Health Stroke Scale (NIHSS), and Barthel Index (BI), Timed Up and Go Test (TUG), Chedoke Arm and Hand Activity Inventory (CAHAI), Symbol Digit Modalities Test, Controlled Oral Word Association tasks, magnetic resonance imaging (MRI), functional magnetic resonance imaging (fMRI), and positron emission tomography (PET) scanning.

In embodiments, therapeutically effective dose achieves remission, cure, response rate, or resolution rate of therapeutically effective dose of between about 10% and about 100% or more. In embodiments, therapeutically effective dose achieves remission, cure, response rate, or resolution rate of a neurological condition between 10% and 100%, such as between 10% and 15%, between 10% and 20%, between 10% and 25%, between 15% and 20%, between 15% and 25%, between 15% and 30%, between 20% and 25%, between 20% and 30%, between 20% and 35%, between 25% and 30%, between 25% and 35%, between 25% and 40%, between 30% and 35%, between 30% and 40%, between 35% and 45%, between 35% and 50%, between 40% and 45%, between 40% and 50%, between 40% and 55%, between 45% and 50%, between 45% and 55%, between 45% and 60%, between 50% and 55%, between 50% and 60%, between 50% and 65%, between 55% and 60%, between 55% and 65%, between 55% and 70%, between 60% and 65%, between 60% and 70%, between 60% and 75%, between 65% and 70%, between 65% and 75%, between 65% and 80%, between 70% and 75%, between 70% and 80%, between 70% and 85%, between 75% and 80%, between 75% and 85%, between 75% and 90%, between 80% and 85%, between 80% and 90%, between 80% and 95%, between 85% and 90%, between 85% and 95%, between 85% and 100%, between 90% and 95%, between 90% and 100%, or between 95% and 100%.

In embodiments, therapeutically effective dose eliminates, reduces, slows, or delays, one or more neurological condition symptoms between 10% and 100%, such as between 10% to about 15%, between 10% and 20%, between 10% and 25%, between 15% and 20%, between 15% and 25%, between 15% and 30%, between 20% and 25%, between 20% and 30%, between 20% and 35%, between 25 and 30%, between 25% and 35%, between 25% and 40%, between 30% and 35%, between 30% and 40%, between 35% and 45%, between 35% and 50%, between 40% and 45%, between 40% and 50%, between 40% and 55%, between 45% and 50%, between 45% and 55%, between 45% and 60%, between 50% and 55%, between 50% and 60%, between 50% and 65%, between 55% and 60%, between 55% and 65%, between 55% and 70%, between 60% and 65%, between 60% and 70%, between 60% and 75%, between 65% and 70%, between 65% and 75%, between 65% and 80%, between 70% and 75%, between 70% and 80%, between 70% and 85%, between 75% and 80%, between 75% and 85%, between 75% and 90%, between 80% and 85%, between 80% and 90%, between 80% and 95%, between 85% and 90%, between 85% and 95%, between 85% and 100%, between 90% and 95%, between 90% and 100%, or between 95% and 100%.

In embodiments, a neurological condition symptom is assessed on the day of treatment, 1 day post treatment, 3 months post treatment, 6 months post treatment, 1 year post treatment and every year thereafter post treatment.

In embodiments, a neurological condition symptom is assessed between 1 day post treatment and 7 days post treatment. In embodiments, symptoms can be assessed between 1 day post treatment and 2 days post treatment, between 1 day post treatment and 3 days post treatment, between 1 day post treatment and 4 days post treatment, between 2 days post treatment and 3 days post treatment, between 2 days post treatment and 4 days post treatment, between 2 days post treatment and 5 days post treatment, between 3 days post treatment and 4 days post treatment, between 3 days post treatment and 5 days post treatment, 3 days post treatment and 6 days post treatment, between 4 days post treatment and 5 days post treatment, between 4 days post treatment and 6 days post treatment, between 4 days post treatment and 7 days post treatment, between 5 days post treatment and 6 days post treatment, between 5 days post treatment and 7 days post treatment, or between 6 days post treatment and 7 days post treatment. In embodiments, symptoms can be assessed between 1 week post treatment and 4 weeks post treatment. In embodiments, symptoms can be assessed between 1 week post treatment and 2 weeks post treatment, between 1 week post treatment and 3 weeks post treatment, between 1 week post treatment and 4 weeks post treatment, between 2 weeks post treatment and 3 weeks post treatment, between 2 weeks post treatment and 4 weeks post treatment, or between 3 weeks post treatment and 4 weeks post treatment. In embodiments, symptoms can be assessed between 1 month post treatment and 12 months post treatment. In embodiments, symptoms can be assessed between 1 month post treatment and 2 months post treatment, between 1 month post treatment and 3 months post treatment, between 1 month post treatment and 4 months post treatment, between 2 months post treatment and 3 months post treatment, between 2 months post treatment and 4 months post treatment, between 2 months post treatment and 5 months post treatment, between 3 months post treatment and 4 months post treatment, between 3 months post treatment and 5 months post treatment, between 3 months post treatment and 6 months post treatment, between 4 months post treatment and 5 months post treatment, between 4 months post treatment and 6 months post treatment, between 4 months post treatment and 7 months post treatment, between 5 months post treatment and 6 months post treatment, between 5 months post treatment and 7 months post treatment, between 5 months post treatment and 8 months post treatment, between 6 months post treatment and 7 months post treatment, between 6 months post treatment and 8 months post treatment, between 6 months post treatment and 9 months post treatment, between 7 months post treatment and 8 months post treatment, between 7 months post treatment and 9 months post treatment, between 7 months post treatment and 10 months post treatment, between 8 months post treatment and 9 months post treatment, between 8 months post treatment and 10 months post treatment, between 8 months post treatment and 11 months post treatment, between 9 months post treatment and 10 months post treatment, between 9 months post treatment and 11 months post treatment, between 9 months post treatment and 12 months post treatment, between 10 months post treatment and 11 months post treatment, between 10 months post treatment and 12 months post treatment, or between 11 months post treatment and 12 months post treatment. In embodiments, symptoms can be assessed between 1 year post treatment and about 20 years post treatment. In embodiments symptoms can be assessed between 1 year post treatment and 5 years post treatment, between 1 year post treatment and 10 years post treatment, between 1 year post treatment and 15 years post treatment, between 5 years post treatment and 10 years post treatment, between 5 years post treatment and 15 years post treatment, between 5 years post treatment and 20 years post treatment, between 10 years post treatment and 15 years post treatment, between 10 years post treatment and 20 years post treatment, or between 15 years post treatment and 20 years post treatment.

As used herein, the term “survival rate” refers to a cohort of subjects in a treatment group still alive after a given period of time after diagnosis of a neurological condition.

In embodiments, therapeutically effective dose achieves increase survival rate of between about 10% and 100% or more. In embodiments, a therapeutically effective dose achieves an increase in survival rate of between 10% and 100%, such as between 10% and 15%, between 10% and 20%, between 10% and 25%, between 15% and 20%, between 15% and 25%, between 15% and 30%, between 20% and 25%, between 20% and 30%, between 20% and 35%, between 25% and 30%, between 25% and 35%, between 25% and 40%, between 30% and 35%, between 30% and 40%, between 35% and 45%, between 35% and 50%, between 40% and 45%, between 40% and 50%, between 40% and 55%, between 45% and 50%, between 45% and 55%, between 45% and 60%, between 50% and 55%, between 50% and 60%, between 50% and 65%, between 55% and 60%, between 55% and 65%, between 55% and 70%, between 60% and 65%, between 60% and 70%, between 60% and 75%, between 65% and 70%, between 65% and 75%, between 65% and 80%, between 70% and 75%, between 70% and 80%, between 70% and 85%, between 75% and 80%, between 75% and 85%, between 75% and 90%, between 80% and 85%, between 80% and 90%, between 80% and 95%, between 85% and 90%, between 85% and 95%, between 85% and 100%, between 90% and 95%, between 90% and 100%, or between 95% and 100%.

As used herein, the term “life expectancy” refers to a period of time a subject is expected to live. In embodiments, life expectancy is determined by gender. In embodiments, life expectancy is determined by genetics. In embodiments, life expectancy is determined by illness. In embodiments, life expectancy is determined by education. In embodiments, life expectancy is determined by mental health. In embodiments, life expectancy is determined by the population of a country.

In embodiments, therapeutically effective dose increases life expectancy of between about 10% and 100% or more. In embodiments, a therapeutically effective dose increases life expectancy of between 10% and 100%, such as between 10% and 15%, between 10% and 20%, between 10% and 25%, between 15% and 20%, between 15% and 25%, between 15% and 30%, between 20% and 25%, between 20% and 30%, between 20% and 35%, between 25% and 30%, between 25% and 35%, between 25% and 40%, between 30% and 35%, between 30% and 40%, between 35% and 45%, between 35% and 50%, between 40% and 45%, between 40% and 50%, between 40% and 55%, between 45% and 50%, between 45% and 55%, between 45% and 60%, between 50% and 55%, between 50% and 60%, between 50% and 65%, between 55% and 60%, between 55% and 65%, between 55% and 70%, between 60% and 65%, between 60% and 70%, between 60% and 75%, between 65% and 70%, between 65% and 75%, between 65% and 80%, between 70% and 75%, between 70% and 80%, between 70% and 85%, between 75% and 80%, between 75% and 85%, between 75% and 90%, between 80% and 85%, between 80% and 90%, between 80% and 95%, between 85% and 90%, between 85% and 95%, between 85% and 100%, between 90% and 95%, between 90% and 100%, or between 95% and 100%.

In embodiments, therapeutically effective dose reduces the amount of atrophy within the brain of a subject in need thereof between about 10% and 100% or more. In embodiments, a therapeutically effective dose reduces the amount of atrophy within the brain of a subject in need thereof between 10% and 100%, such as between 10% and 15%, between 10% and 20%, between 10% and 25%, between 15% and 20%, between 15% and 25%, between 15% and 30%, between 20% and 25%, between 20% and 30%, between 20% and 35%, between 25% and 30%, between 25% and 35%, between 25% and 40%, between 30% and 35%, between 30% and 40%, between 35% and 45%, between 35% and 50%, between 40% and 45%, between 40% and 50%, between 40% and 55%, between 45% and 50%, between 45% and 55%, between 45% and 60%, between 50% and 55%, between 50% and 60%, between 50% and 65%, between 55% and 60%, between 55% and 65%, between 55% and 70%, between 60% and 65%, between 60% and 70%, between 60% and 75%, between 65% and 70%, between 65% and 75%, between 65% and 80%, between 70% and 75%, between 70% and 80%, between 70% and 85%, between 75% and 80%, between 75% and 85%, between 75% and 90%, between 80% and 85%, between 80% and 90%, between 80% and 95%, between 85% and 90%, between 85% and 95%, between 85% and 100%, between 90% and 95%, between 90% and 100%, or between 95% and 100%.

In embodiments, the amount of atrophy within the brain of a subject in need is assessed on the day of treatment, 1 day post treatment, 3 months post treatment, 6 months post treatment, 1 year post treatment and every year thereafter post treatment.

In embodiments, the amount of atrophy within the brain of a subject in need is assessed between 1 day post treatment and 7 days post treatment. In embodiments, symptoms can be assessed between 1 day post treatment and 2 days post treatment, between 1 day post treatment and 3 days post treatment, between 1 day post treatment and 4 days post treatment, between 2 days post treatment and 3 days post treatment, between 2 days post treatment and 4 days post treatment, between 2 days post treatment and 5 days post treatment, between 3 days post treatment and 4 days post treatment, between 3 days post treatment and 5 days post treatment, 3 days post treatment and 6 days post treatment, between 4 days post treatment and 5 days post treatment, between 4 days post treatment and 6 days post treatment, between 4 days post treatment and 7 days post treatment, between 5 days post treatment and 6 days post treatment, between 5 days post treatment and 7 days post treatment, or between 6 days post treatment and 7 days post treatment. In embodiments, symptoms can be assessed between 1 week post treatment and 4 weeks post treatment. In embodiments, symptoms can be assessed between 1 week post treatment and 2 weeks post treatment, between 1 week post treatment and 3 weeks post treatment, between 1 week post treatment and 4 weeks post treatment, between 2 weeks post treatment and 3 weeks post treatment, between 2 weeks post treatment and 4 weeks post treatment, or between 3 weeks post treatment and 4 weeks post treatment. In embodiments, symptoms can be assessed between 1 month post treatment and 12 months post treatment. In embodiments, symptoms can be assessed between 1 month post treatment and 2 months post treatment, between 1 month post treatment and 3 months post treatment, between 1 month post treatment and 4 months post treatment, between 2 months post treatment and 3 months post treatment, between 2 months post treatment and 4 months post treatment, between 2 months post treatment and 5 months post treatment, between 3 months post treatment and 4 months post treatment, between 3 months post treatment and 5 months post treatment, between 3 months post treatment and 6 months post treatment, between 4 months post treatment and 5 months post treatment, between 4 months post treatment and 6 months post treatment, between 4 months post treatment and 7 months post treatment, between 5 months post treatment and 6 months post treatment, between 5 months post treatment and 7 months post treatment, between 5 months post treatment and 8 months post treatment, between 6 months post treatment and 7 months post treatment, between 6 months post treatment and 8 months post treatment, between 6 months post treatment and 9 months post treatment, between 7 months post treatment and 8 months post treatment, between 7 months post treatment and 9 months post treatment, between 7 months post treatment and 10 months post treatment, between 8 months post treatment and 9 months post treatment, between 8 months post treatment and 10 months post treatment, between 8 months post treatment and 11 months post treatment, between 9 months post treatment and 10 months post treatment, between 9 months post treatment and 11 months post treatment, between 9 months post treatment and 12 months post treatment, between 10 months post treatment and 11 months post treatment, between 10 months post treatment and 12 months post treatment, or between 11 months post treatment and 12 months post treatment. In embodiments, symptoms can be assessed between 1 year post treatment and about 20 years post treatment. In embodiments symptoms can be assessed between 1 year post treatment and 5 years post treatment, between 1 year post treatment and 10 years post treatment, between 1 year post treatment and 15 years post treatment, between 5 years post treatment and 10 years post treatment, between 5 years post treatment and 15 years post treatment, between 5 years post treatment and 20 years post treatment, between 10 years post treatment and 15 years post treatment, between 10 years post treatment and 20 years post treatment, or between 15 years post treatment and 20 years post treatment.

Non-limiting examples of tests to evaluate the amount of atrophy within the brain of a subject in need include Nissle staining, MRI, functional magnetic resonance fMRI, and PET scanning.

In embodiments, the disclosure includes an article of manufacture. In certain aspects, the article of manufacture includes a closed or sealed package that contains one or a combination of the compounds described herein, such as in separate tablets, capsules or the like. The package can comprise one or more containers, such as closed or sealed vials, bottles, blister (bubble) packs, or any other suitable packaging for the sale, or distribution, or use of pharmaceutical agents. Thus, the package can contain pharmaceutical compositions which comprise insulin, forskolin, and which may further comprise VC and/or Cri. Any one or all of these compounds can be included, and each can be provided separately or in combination with one or more of the others in the same or distinct dosage formulations so that they can be delivered concurrently, or sequentially.

In addition to the pharmaceutical compositions, the package may contain printed information. The printed information can be provided on a label, or on a paper insert, or printed on the packaging material itself. The printed information can include information that identifies the active agents in the package, the amounts and types of inactive ingredients, an indication of what condition(s) the pharmaceutical composition(s) is intended to treat, and instructions for taking the pharmaceutical composition, such as the number of doses to take over a given period of time, the order to take the compositions, and the like. In embodiments the disclosure includes a pharmaceutical composition of the invention packaged in a packaging material and identified in print, on or in the packaging material, that the composition is for use in the treatment or prophylaxis of any disease, condition or disorder that is related to a deterioration of neurons, an insufficiency of neurons, or a defect in the function of neurons. In embodiments, instead of a pharmaceutical composition, the disclosure includes a nutraceutical formulation(s), and the printed material provides information about use of such a formulation(s) for improving neurological condition symptoms. Non-limiting examples of neurological conditions symptoms include cognitive function, memory, motor function, overall well-being, or the like.

The following Example is provided to illustrate the invention, but is not intended to be limiting in any way.

### Example

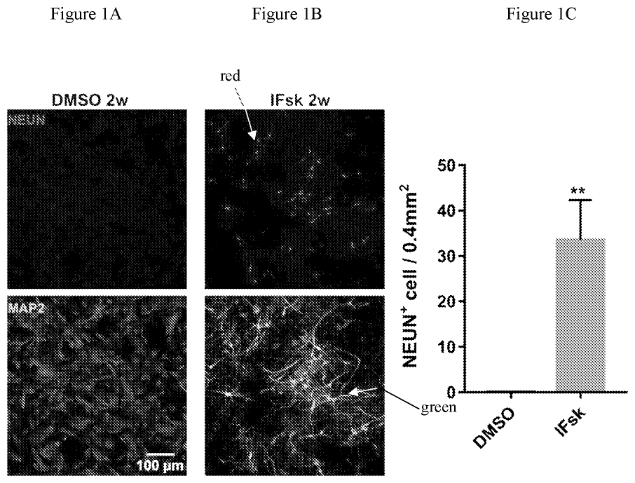

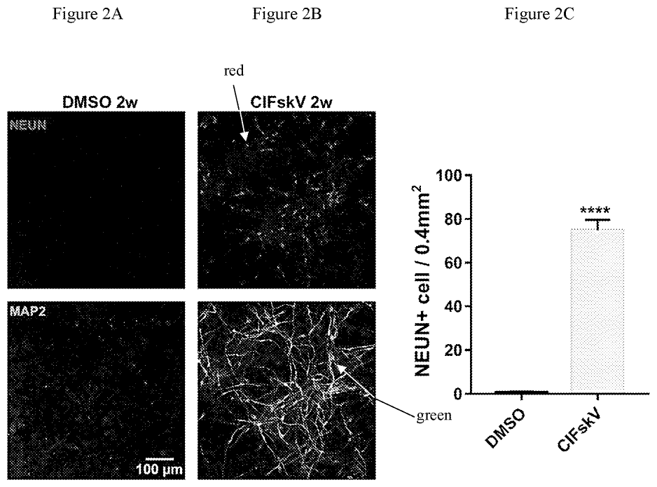

This Example demonstrates, as shown in FIG. 1, that a combination of forskolin and insulin is sufficient to reprogram human astrocytes into neurons. Further, as shown in FIG. 2, adding VC and Crizotinib to the combination of forskolin and insulin improves neuronal generation. The results in FIGS. 1 and 2 were produced using the following materials and methods.

Cell culture. Human astrocytes (ScienCell, 1800) were cultured for at least 10 passages before experiment in the medium containing Dulbecco's Modified Eagle Medium/Nutrient Mixture F-12 (DMEM/F12 (Gibco), B27 supplements (Gibco), 10% Fetal bovine serum (FBS) (Gibco), 3.5 mM Glucose (Sigma), 10 ng/mL epidermal growth factor (EGF, Alomone labs), 10 ng/mL fibroblast growth factor 2 (FGF2, Alomone labs), and penicillin/streptomycin (Gibco). Human astrocytes were then seeded onto poly-D-Lysine (Sigma) coated glass coverslip in 24-well plates (BD Biosciences) until cell density reached around 90% confluence. Half of the medium was changed to conversion medium, which contains only DMEM/F12 and penicillin/streptomycin.

Compound Treatment

0.25 μM Crizotinib (C), 50 mg/L insulin (I), 10 μM forskolin (Fsk), and 50 μg/mL Vitamin C (VC) were provided to human astrocytes in the combination of either IFsk or CIFskV together. A corresponding amount of DMSO as was used to dissolve Crizotinib and forskolin served as control. Drug treatment lasted for 12 days with drugs being refreshed every 3 days. At day 12, conversion medium and drugs were changed to neuron differentiation medium that contained DMEM/F12, B27 supplement, N2 supplement (Gibco), 0.5% FBS, 5 mg/mL VC, 1 μM Y-27632 (Tocris), and penicillin/streptomycin.

Immunocytochemistry

Cells were first fixed on coverslips with 4% Paraformaldehyde (PFA) (Alfa Aesar) for 12 mins, then washed with phosphate buffered saline (PBS for three times. After fixation, cells were blocked with 2.5% normal donkey serum (Jackson ImmunoResearch), 2.5% normal goat serum (Jackson ImmunoResearch), 0.1% Triton X-100 (Fisher Scientific) in PBS for one hour at room temperature. During blocking, the plate holding coverslips were placed on a slow-speed shaker. After blocking, primary antibodies polyclonal anti-NEUN (rabbit, 1:1000, Millipore, ABN78) and polyclonal anti-MAP2 (Chicken, 1:2000, Abcam, AB5392) in the same blocking buffer were applied on coverslips that hold cells. Primary antibody incubation was performed at 4° C. overnight. The next day, primary antibodies were washed away with PBS. Cells were then incubated with the corresponding secondary antibodies (1:800, Molecular Probes) for another one hour in room temperature, after which, secondary antibodies were washed away with PBS for three times. Cells on coverslips were counted on glass slides. Coverslips with cells were mounted on glass slides with mounting solution containing DAPI (Invitrogen). Images of immunostaining result were taken with confocal microscopes ZEISS LSM800.

In an embodiment, the disclosure includes a method for generating neurons comprising contacting glial cells with a combination of compounds comprising forskolin and insulin, wherein the combination of forskolin and insulin is sufficient to generate the neurons from the glial cells.

In an embodiment, the method further comprises contacting the glial cells with Vitamin C (VC), Crizotinub (Cri), or a combination thereof. In an embodiment, the method further comprises contacting the glial cells with a combination of the VC and the Cri to enhance neuronal generation relative to neuronal generation produced by contacting the glial cells with a combination of forskolin and insulin alone. In an embodiment, the forskolin is comprised by at least one pharmaceutical formulation.

In an embodiment, the insulin is comprised by at least one pharmaceutical formulation. In embodiments of the foregoing, the combination of forskolin and insulin are comprised by the same pharmaceutical formulation. In embodiments of the foregoing, the forskolin and insulin are active ingredients in the at least one pharmaceutical formulation. In embodiments of the foregoing, the forskolin and insulin are the only active ingredients in the at least one pharmaceutical formulation. In embodiments of the foregoing, the VC is comprised by the at least one pharmaceutical formulation. In embodiments of the foregoing, the Cri is comprised by the at least one pharmaceutical formulation. In embodiments of the foregoing, the VC and the Cri are comprised by the at least one pharmaceutical formulation. In embodiments of the foregoing, the forskolin, the insulin, the VC and the Cri are comprised by the at least one pharmaceutical formulation.

In embodiments of the foregoing, the glial cells are present in a subject in need of neuronal generation. In embodiments of the foregoing, the subject in need of the neuronal generation has a neurodegenerative disorder. In embodiments of the foregoing, the subject is in need of treatment for has a neuronal condition selected from the group consisting of Alzheimer's disease, another conditions which presents with dementia, Chronic Traumatic Encephalopathy (CTE), Parkinson's disease, Huntington's disease, multiple sclerosis, spinal cord injury, spinal muscular atrophy, Amyotrophic lateral sclerosis (ALS), and stroke. In an embodiment, the subject has Alzheimer's disease. In an embodiment, the subject has Huntington's disease.

In an embodiment the disclosure provides a pharmaceutical composition comprising a combination of forskolin and insulin, and the pharmaceutical composition optionally further comprising one or both of Vitamin C (VC) and Crizotinub (Cri). In an embodiment the disclosure provides a pharmaceutical a comprising the forskolin, the insulin, the VC, and the Cri. In an embodiment, the forskolin and the insulin are active ingredients and are the only active ingredients in a pharmaceutical formulation. In an embodiment, the forskolin, the insulin, the VC, and the Cri are active ingredients and are the only active ingredients in a pharmaceutical formulation.

In another embodiment the disclosure provides an article of manufacture comprising a pharmaceutical composition comprising a combination of forskolin and insulin, the article of manufacture further comprising printed material providing an indication that a combination is for use in treating a condition associated with a need for functional neurons. In an embodiment, in the article of manufacture, the forskolin and the insulin are active ingredients and are the only active ingredients in a pharmaceutical formulation. In an embodiment, in the article of manufacture, the pharmaceutical formulation further comprises one of Vitamin C (VC) or Crizotinub (Cri). In an embodiment, in the article of manufacture, the VC and the Cri are present in the pharmaceutical formulation, wherein the VC and the Cri are active ingredients, and wherein the forskolin, the insulin, the VC and the Cri are the only active ingredients in the pharmaceutical formulation.

While the invention has been described through specific embodiments, routine modifications will be apparent to those skilled in the art and such modifications are intended to be within the scope of the present invention.

What is claimed is:

1. A method for generating neurons comprising contacting glial cells with a combination of compounds comprising forskolin and insulin, wherein the combination of forskolin and insulin is sufficient to generate the neurons from the glial cells.

2. The method of claim 1, further comprising contacting the glial cells with Vitamin C (VC), Crizotinub (Cri), or a combination thereof.

3. The method of claim 2, comprising contacting the glial cells with a combination of the VC and the Cri to enhance neuronal generation relative to neuronal generation produced by contacting the glial cells with a combination of forskolin and insulin alone.

4. The method of claim 1, wherein the forskolin is comprised by at least one pharmaceutical formulation.

5. The method of claim 1 or 4, wherein the insulin is comprised by at least one pharmaceutical formulation.

6. The method of any one of claims 1, 4, and 5, wherein the combination of forskolin and insulin are comprised by the same pharmaceutical formulation.

7. The method of any one of claims 1, and 4 to 6, wherein the forskolin and insulin are active ingredients in the at least one pharmaceutical formulation.

8. The method of any one of claims 1, and 4 to 7, wherein the forskolin and insulin are the only active ingredients in the at least one pharmaceutical formulation.

9. The method of any one of claims 1, and 4 to 8, wherein the VC is comprised by the at least one pharmaceutical formulation.

10. The method of any one of claims 1, and 4 to 9, wherein the Cri is comprised by the at least one pharmaceutical formulation.

11. The method of any one of claims 1, and 4 to 10, wherein the VC and the Cri are comprised by the at least one pharmaceutical formulation.

12. The method of any one of claims 1, and 4 to 11, wherein the forskolin, the insulin, the VC and the Cri are comprised by the at least one pharmaceutical formulation.

13. The method of any one of claims 1, and 4 to 12, wherein the forskolin, the insulin, the VC and the Cri are active ingredients and are the only active ingredients in the at least one pharmaceutical formulation.

14. The method of any one of claims 1-13, wherein the glial cells are present in a subject in need of neuronal generation.

15. The method of claim 14, wherein the subject in need of the neuronal generation has a neurodegenerative disorder.

16. The method of claim 14, wherein the subject is in need of treatment for has a neuronal condition selected from the group consisting of Alzheimer's disease, another conditions which presents with dementia, Chronic Traumatic Encephalopathy (CTE), Parkinson's disease, Huntington's disease, multiple sclerosis, spinal cord injury, spinal muscular atrophy, Amyotrophic lateral sclerosis (ALS), and stroke.

17. The method of claim 16, wherein the subject has Alzheimer's disease.

18. The method of claim 16, wherein the subject has Huntington's disease.

19. A pharmaceutical composition comprising a combination of forskolin and insulin, the pharmaceutical composition optionally further comprising one or both of Vitamin C (VC) and Crizotinub (Cri).

20. The pharmaceutical composition of claim 19, comprising the forskolin, the insulin, the VC, and the Cri.

21. The pharmaceutical composition of claim 19, wherein the forskolin and the insulin are active ingredients and are the only active ingredients in a pharmaceutical formulation.

22. The pharmaceutical composition of claim 19, the forskolin, the insulin, the VC, and the Cri are active ingredients and are the only active ingredients in a pharmaceutical formulation.

23. An article of manufacture comprising a pharmaceutical composition comprising a combination of forskolin and insulin, the article of manufacture further comprising printed material providing an indication that a combination is for use in treating a condition associated with a need for functional neurons.

24. The article of manufacture of claim 23, wherein the forskolin and the insulin are active ingredients and are the only active ingredients in a pharmaceutical formulation.

25. The article of manufacture of claim 24, wherein the pharmaceutical formulation further comprises one of Vitamin C (VC) or Crizotinub (Cri).

26. The article of manufacture of claim 25, wherein the VC and the Cri are present in the pharmaceutical formulation, wherein the VC and the Cri are active ingredients, and wherein the forskolin, the insulin, the VC and the Cri are the only active ingredients in the pharmaceutical formulation.
